SEQUENCE LISTING (4 sequences)

SEQ ID NO: 1 - protein, 284 aa, Artificial Sequence
  sGal-9
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQHPPYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 2 - protein, 146 aa, Artificial Sequence
  CCRD
TPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 3 - protein, 10 aa, Artificial Sequence
  CCRD 1-10
TPAIPPMMYP

SEQ ID NO: 4 - protein, 283 aa, Artificial Sequence
  sGal-9_Met_del
AFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQHPPYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

(19) United States
(12) Patent Application Publication
(10) Pub. No.: US 2023/0414712 A1
(43) Pub. Date: Dec. 28, 2023

(54) PHARMACEUTICAL COMPOSITION FOR PREVENTING OR TREATING CANCER COMPRISING RECOMBINANT STABILIZED GALECTIN 9 PROTEIN

(71) Applicant: GBIOLOGICS INC., Seongnam-si, Gyeonggi-do (KR)

(72) Inventor: David SONG, Seongnam-si, Gyeonggi-do (KR)

(21) Appl. No.: 18/037,602

(22) PCT Filed: Nov. 11, 2021
(86) PCT No.: PCT/KR2021/016449

§ 371 Date: May 18, 2023

(30) Foreign Application Priority Data
Nov. 18, 2020 (KR) 10-2020-0154641

(51) Int. Cl. A61K 38/17; A61K 31/7068; A61P 35/00; A61K 31/517

(52) U.S. Cl. CPC A61K 38/1732; A61K 31/517; A61P 35/00; A61K 31/7068

(57) ABSTRACT

The present invention relates to a pharmaceutical composition for preventing or treating cancer, comprising a recombinant stabilized galectin-9 protein, and it was confirmed that as a result of treating cancer cells with the recombinant stabilized galectin-9 protein, the effects of inhibiting the cell growth of the cancer cells and inducing cell death were exhibited; as a result of administering the recombinant stabilized galectin-9 protein to a cancer-induced animal model, the effects of reducing the size of cancer tissue and increasing viability without side effects such as weight loss were exhibited; and, when a complex, mixture or combination of the protein and an anticancer drug is administered, synergistic anticancer activity was exhibited, and thus the pharmaceutical composition comprising a recombinant stabilized galectin-9 protein as an active ingredient, according to the present invention, can be effectively used for prevention or treatment of cancer.

### TECHNICAL FIELD

The present invention relates to a recombinant stabilized galectin-9 protein and its use, specifically, a pharmaceutical composition for prevention or treatment of cancer comprising the protein as an active ingredient.

### BACKGROUND ART

It has been found that there are animal lectins in vivo that specifically recognize glycochains with a β-galactoside structure, and so far at least 14 genes have been identified. Galectin is classified as prototype, chimera, and tandem repeat type based on its structure.

Galectin-9, one of the tandem repeat-type galectins, consists of two glycohydrate recognition sites (carbohydrate recognition domain: CRD) and a link peptide region connecting them each other, and so N-terminal Carbohydrate Recognition Domain (NCRD) and C-terminal Carbohydrate Recognition Domain (CCRD) is linked by the link peptide region, and its multifaceted activities have been reported up to the present. As for T cells, galectin-9 binds to Tim-3 to induce apoptosis of Tim-3-positive Th1 cells and inhibits autoimmune inflammation by inhibiting the excess Th1 response. It also reduces Th17 cells, which are one of the causes or exacerbating factors of various incurable diseases such as autoimmune diseases, allergies and cancers expressed by Tim-3.

To utilize the galectin-9 as an actual therapeutic agent, the research involved cleaving the linker peptides of galectin-9 to produce galectin-9 variants with protein-degrading enzyme resistance, such as G9Null (non-patent literature 1) is being continued to address issues such as 1) protein-degrading enzyme sensitivity, 2) low solubility, and 3) low yields of galectin-9.

On the other hand, cancer is a disease caused by an abnormal growth of uncontrolled cells which can spread in contact with tissues or other parts of the body, and cancer cells may form solid tumors in which the cancer cells clumped together or may exist as dispersed cells as in leukemia. Normal cells differentiate until they mature and then replace damaged or dead cells as needed, but cancer cells constantly differentiate and eventually push nearby cells away and spread to other parts, which is called malignant. Malignant tumor cells spread through the bloodstream or lymphatic system to other parts of the body, where they multiply and form new tumors.

In addition, despite the development of various treatment options, cancer still poses a serious threat to human health worldwide. Currently, cancer treatment is mainly conducted by surgical procedures, radiotherapy, hormone therapy, and chemotherapy. Among them, chemotherapy is a method of treating cancer directly or relieving symptoms using one or more anti-cancer drugs. The chemotherapy is known to be one of the most effective methods of treating cancer patients, but the continued administration of anti-cancer drugs has been cited as a cause of failure of the chemotherapy. This is due to the cells acquire resistance to anti-cancer drugs, and if they acquire resistance to one anti-cancer agent, they can also obtain Multidrug Resistance for anti-cancer drugs of different structures.

In addition, RAS protein is a Small GTPase protein that plays an important role in the signaling system associated with the differentiation, proliferation and survival of cells. The RAS family is known for its three isoforms of HRAS, NRAS, and KRAS, and is well known as the oncogene, where mutations are found in several carcinomas. In particular, high frequency of RAS mutations have been reported in lung cancer, colorectal and pancreatic cancers with high fatality rates, and about 85% of RAS-driven cancers are known to be due to KRAS mutations. When mutation occurs at the active site of KRAS, GTP hydrolysis by GTPase-activating proteins (GAPS) does not occur smoothly, resulting in an increase in the cellular level of GTP-bound RAS protein, and abnormally activation of the sub signaling system to cause proliferation of cancer cells.

Accordingly, as a result of the efforts of the present inventors to develop anticancer agents, it is confirmed that recombinant stabilized galectin-9 protein with mutations and substitutions in the C-terminal domain (CCRD) of the two carbohydrate chain recognition sites and in the linker peptides of the wild-type Galectin-9 protein have an effect of reducing the cancer tissue size and increasing the survival rate of mice induced with cancer, as well as observing a significant increase in the anticancer effect when combining the recombinant stabilized galectin-9 protein with anticancer agent, and so it can be used as a complex, mixed or combined anticancer agent and the present invention was completed based thereon.

### CITATION LIST

### Patent Literature

KR Patent No. 10-1222281
JP Patent No. 5888761
U.S. Pat. No. 9,908,921

### Non Patent Literature

Nishi, N et al., FEBS Lett. 2005 Apr. 11; 579(10):2058-64.
Hobbs, G. A, et al., RAS isoforms and mutations in cancer at a glance. J Cell Sci 2016, 129(7), 1287-92

### SUMMARY OF INVENTION

### Technical Problem

The object of the present invention is to provide a recombinant stabilized galectin-9 protein and a pharmaceutical composition for prevention or treatment of cancer comprising a recombinant stabilized galectin-9 protein.

### Solution to Problem

To achieve the object of the present invention, the present invention provides a pharmaceutical composition for preventing or treating cancer comprising a recombinant stabilized galectin-9 protein having an amino acid sequence represented by SEQ ID NO: 1 or a polynucleotide encoding the same as an active ingredient; a method of preventing or treating cancer comprising administering to a subject a recombinant stabilized galectin-9 protein having an amino acid sequence represented by SEQ ID NO: 1 or a polynucleotide encoding the same in a pharmaceutically effective amount; and use of pharmaceutical composition comprising a recombinant stabilized galectin-9 protein having an amino acid sequence represented by SEQ ID NO: 1 or a polynucleotide encoding the same as an active ingredient for use in preventing or treating cancer.

In addition, the present invention provides a pharmaceutical composition for preventing or treating cancer comprising a recombinant stabilized galectin-9 protein having an amino acid sequence represented by SEQ ID NO: 1 or a polynucleotide encoding the same and an anti-cancer agent as active ingredients of a complex, mixed, or combination agent; a method of preventing or treating cancer comprising administering a formulation comprising a recombinant stabilized galectin-9 protein having an amino acid sequence represented by SEQ ID NO: 1 or a polynucleotide encoding the same in a pharmaceutically effective amount and a formulation comprising an anticancer agent, to a subject in a complex, mixed, or combination manner; and use of pharmaceutical composition comprising a recombinant stabilized galectin-9 protein having an amino acid sequence represented by SEQ ID NO: 1 or a polynucleotide encoding the same, and anticancer agent in active ingredients of a complex, mixed, or combination agent for preventing or treating cancer.

### Advantageous Effects of Invention

The recombinant stabilized galectin-9 protein of the present invention induces cell growth inhibition and apoptosis of cancer cells, and exhibits the effect of reducing cancer tissue size and increasing survival rate of cancer-causing mice without side effects such as weight loss, and show synergistic anticancer activity by administering in complex, mixed or combination with anticancer drug, and so it can be used as an active ingredient of pharmaceutical composition for cancer prevention or treatment.

### BRIEF DESCRIPTION OF DRAWINGS

FIGS. 1A and 1B are diagrams for confirming the inhibition of cell growth (FIG. 1A) and induction of apoptosis of cell death (FIG. 1B) by recombinant stabilized galectin-9 protein (seal-9) in various cancer cell lines with KRAS wild type or KRAS gene mutation.

FIG. 2 is a diagram for confirming the inhibition of cell proliferation by seal-9 in DLD-1 cells, a colorectal cancer cell line with KRAS gene mutation.

FIG. 3 is a diagram for confirming the changes in cell growth when seal-9 and the EGFR inhibitor Erlotinib were co-treated in DLD-1 cells, a colorectal cancer cell line with KRAS gene mutation.

FIG. 4 is a diagram for confirming the weight changes in a KRAS gene mutant pancreatic cancer-induced mouse model treated with seal-9 and/or gemcitabine:
(1) Pink: Results of measuring weight (g) change when PBS (control) was administered alone;
(2) Orange: Results of measuring weight (g) change when 0.5 mg/kg of seal-9 was administered alone;
(3) Dark green: Results of measuring weight (g) change when 5 mg/kg of seal-9 was administered alone;
(4) Green: Results of measuring weight (g) change when 50 mg/kg of gemcitabine was administered alone;
(5) Blue: Results of measuring weight (g) change when 5 mg/kg of seal-9 and 50 mg/kg of gemcitabine were administered in combination.

FIG. 5 is a diagram for confirming the changes in tumor size in a KRAS gene mutant pancreatic cancer-induced mouse model after treatment with recombinant stabilized galectin-9 protein (seal-9) and/or gemcitabine:
(1) Pink: Results of measuring changes in tumor size (mm³) when PBS (control) was administered alone;
(2) Orange: Results of measuring changes in tumor size (mm³) when 0.5 mg/kg of seal-9 was administered alone;
(3) Dark green: Results of measuring changes in tumor size (mm³) when 5 mg/kg of seal-9 was administered alone;
(4) Green: Results of measuring changes in tumor size (mm³) when 50 mg/kg of gemcitabine was administered alone;
(5) Blue: Results of measuring changes in tumor size (mm³) when 5 mg/kg of seal-9 and 50 mg/kg of gemcitabine were administered in combination.

FIG. 6 is a diagram for confirming the changes in survival rate of a KRAS gene mutant pancreatic cancer-induced mouse model treated with seal-9 and/or gemcitabine:
(1) Pink: Results of measuring survival rate (%) when PBS (control) was administered alone;
(2) Orange: Results of measuring survival rate (%) when 0.5 mg/kg of seal-9 was administered alone;
(3) Dark green: Results of measuring survival rate (%) when 5 mg/kg of seal-9 was administered alone;
(4) Green: Results of measuring survival rate (%) when 50 mg/kg of gemcitabine was administered alone;
(5) Blue: Results of measuring survival rate (%) when 5 mg/kg of seal-9 and 50 mg/kg of gemcitabine were administered in combination.

### DESCRIPTION OF EMBODIMENTS

Hereinafter, the embodiments of the present invention will be described in detail so that it can be easily practiced by those of ordinary skill in the art to which the present invention belongs. Embodiments of the present invention are provided to those of average knowledge in the art to be fully explained of the invention. Accordingly, embodiments of the present invention may be modified in a variety of different forms, and the scope of the present invention is not limited to the embodiments described below.

Throughout the entire specification of the present invention, when a portion is described as “including” a particular component, it means that the portion may also include other components rather than excluding other components, unless specifically stated to the contrary.

The present invention provides a pharmaceutical composition for preventing or treating cancer comprising a recombinant stabilized galectin-9 protein having an amino acid sequence represented by SEQ ID NO: 1 or a polynucleotide encoding the same as an active ingredient; a method of preventing or treating cancer comprising administering to a subject a recombinant stabilized galectin-9 protein having an amino acid sequence represented by SEQ ID NO: 1 or a polynucleotide encoding the same in a pharmaceutically effective amount; and use of pharmaceutical composition comprising a recombinant stabilized galectin-9 protein having an amino acid sequence represented by SEQ ID NO: 1 or a polynucleotide encoding the same as an active ingredient for use in preventing or treating cancer.

In the present invention, the recombinant stabilized galectin-9 protein can have effects of reducing the size of cancerous tissue and increasing survival rate without side effects such as weight loss.

The term, “prevention/preventing” as used herein, refers to any action that inhibits the onset or delays the invention by administration of the composition.

The term “treatment/treating” as used herein, refers to any action in which the symptoms of the disease are improved or beneficially altered by administration of the composition.

In the present invention, the recombinant stabilized galectin-9 protein is a protein having a more stable molecular structure for the protease while maintaining the glycosyncognistic activity of wild type Galectin-9.

Specifically, the recombinant stabilized galectin-9 protein is a recombinant protein prepared by modifying a link region connecting two carbohydrate recognition domains (CRDs) of wild-type galectin-9 having the structure of NCRD-linker-CCRD and a C-terminal carbohydrate recognition domain (CCRD). More specifically, the recombinant stabilized galectin-9 protein is obtained by deleting all peptides of the linker region, deleting the amino acid sequence at positions 1 to 10 (SEQ ID NO: 3) in CCRD (SEQ ID NO: 2), and substituting Ala (Alanine; A) at position 13 with Pro (Proline; P), may include the amino acid sequence represented by SEQ ID NO: 1, may include an amino acid sequence having sequence homology of 75% or more, preferably 80% or more, more preferably 90% or more with the amino acid sequence represented by SEQ ID NO: 1, and may further include targeting sequences, tags, labeled residues, and an amino acid sequence prepared for a specific purpose to increase half-life or peptide stability.

In addition, the recombinant stabilized galectin-9 protein may comprise the deletion of the first amino acid residue from the N-terminus of the amino acid sequence represented by SEQ ID NO: 1, and it may consist of an amino acid sequence specifically represented by SEQ ID NO: 4.

As used herein, the term “polynucleotide” refers to a polymer to which nucleotides are bound, and serves to transmit genetic information. For the purposes of the present invention, polynucleotide encodes the recombinant protein of SEQ ID NO: 1 and may include sequence having sequence homology of 75% or more, preferably 85% or more, more preferably 90% or more, and most preferably 95% or more with the polynucleotide sequence for encoding the recombinant protein.

As used herein, the term “homology” is intended to indicate a degree of similarity to a wild type amino acid sequence or a polynucleotide sequence, the comparison of such homology can be performed using a comparison program well known in the art, and homology between two or more sequences can be calculated as a percentage (%).

In the present invention, the cancer comprises (A) (1) orthogonal conduit carcinoma (DCIS) (cotton carcinoma, ideogram, nipple, micropapilloma), invasive conduit carcinoma (IDC), tubular carcinoma, mucus (colloidal) carcinoma, papillary carcinoma, incarnate carcinoma and conduit carcinoma, including inflammatory carcinoma; (2) lobular carcinoma, including orthogonal lobular carcinoma (LCIS) and invasive lobular carcinoma; and (3) breast cancer, including paget's disease of the nipples; (B) (1) cervical epithelial tumors (Grade I), cervical epithelial tumors (Class II), intracervical epithelial tumors (Class III) (orthogonal squamous cell carcinoma), keratinized squamous cell carcinoma, non-keratinous squamous cell carcinoma, wart-shaped carcinoma, orthogonal adenocarcinoma, orthogonal adenocarcinoma, endometrial adenocarcinoma, transparent cell adenocarcinoma, adenoepithelial carcinoma, adenocarcinoma, adenocarcinoma, small cell carcinoma and undifferentiated carcinoma; (2) Cancer of the uterine body, including endometrial carcinoma, adenocarcinoma, adenocarcinoma (adenocarcinoma with squamous epithelium), adenocarcinoma (mixed adenocarcinoma and squamous cell carcinoma, mucous adenocarcinoma, serous adenocarcinoma, transparent cell adenocarcinoma, squamous cell adenocarcinoma and undifferentiated adenocarcinoma; (3) Cancer of the ovaries, including serous cystadenoma, serous cystadenoma, mucous cystadenoma, mucous cyst adenoma, endometrial tumor, endometrial adenocarcinoma, transparent cell tumor, transparent cell cyst adenoma and unclassified tumor; (4) vaginal cancers, including squamous cell carcinoma and adenocarcinoma; And (5) tumors in the vulva epithelial (Class I), tumors in the vulva epithelial (Class II), tumors in the vulva epithelial (Class III) (orthogonal squamous cell carcinoma); Cancers of the female reproductive system, including cancer of the vulva, including squamous cell carcinoma, wart-shaped carcinoma, paget's disease of the pubic gland, adenocarcinoma (NOS), basal cell carcinoma (NOS) and Bartholin adenocarcinoma; (C) (1) cancer of the penis, including squamous cell carcinoma; (2) Cancer of the prostate, including adenocarcinoma of the prostate, sarcoma and transitional cell carcinoma; (3) Cancers of the male reproductive system, including cancers of the testicles, including normal-hematoma tumors, abnormal hematoma tumors, teratomas, embryonic carcinoma, yolk cyst tumors and chorionic carcinoma; (D) sarcoma (angiosarcoma, fibrosarcoma, rhabdomyosarcoma, liposarcoma), myxoma, rhabdomyomyoma, fibroma, lipoma and teratoma of the heart system; (E) cancers of the respiratory system, including squamous cell carcinoma of the larynx, primary pleural mesothelioma and squamous cell carcinoma of the pharynx; (F) squamous cell carcinoma (epidermal carcinoma), variants of squamous cell carcinoma, spindle cell carcinoma, small cell carcinoma, carcinoma, carcinoma, carcinoma of other cells, carcinoma of intermediate cell type, complex oat cell carcinoma, adenocarcinoma, granular adenocarcinoma, papillary adenocarcinoma, bronchial alveolar carcinoma, mucous forming solid carcinoma, megaloblastic carcinoma, cytocarcinoma, transparent cell carcinoma, and cancer of the lungs, including sarcoma; (G) (1) cancer of the Vater bulge, including primary adenocarcinoma, carcinoid tumors and lymphoma; (2) Cancer of the tract, including adenocarcinoma, squamous cell carcinoma and melanoma; (3) Cancer of the extrahepatic bile ducts, including orthogonal carcinoma, adenocarcinoma, adenocarcinoma, intestinal type, mucous adenocarcinoma, transparent cell adenocarcinoma, ring cell carcinoma, adenoepithelial carcinoma, squamous cell carcinoma, small cell (oats) carcinoma, undifferentiated carcinoma, carcinoma (NOS), sarcoma and carcinoid tumors; (4) Orthogonal adenocarcinoma, adenocarcinoma, mucous adenocarcinoma (colloidal type; mucous carcinoma greater than 50%), ring cell carcinoma (ring cells greater than 50%), squamous cell (epidermis-shaped) carcinoma, adenoepithelial carcinoma, small cell (oat cell) carcinoma, undifferentiated carcinoma, carcinoma (NOS), sarcoma, lymphoma and cancer of the colon and rectum, including carcinoma; (5) Cancer of the esophagus, including squamous cell carcinoma, adenocarcinoma, smooth myoma and lymphoma; (6) Cancer of the gallbladder, including adenocarcinoma, adenocarcinoma, bowel type, adenoepithelial carcinoma, orthogonal carcinoma, carcinoma (NOS), transparent cell adenocarcinoma, mucous adenocarcinoma, papillary adenocarcinoma, ring cell carcinoma, small cell (oat cell) carcinoma, squamous cell carcinoma and undifferentiated carcinoma; (7) Cancer of the lips and oral cavity, including squamous cell carcinoma; (8) Cancer of the liver, including liver cancer (hepatocellular carcinoma), bile duct carcinoma, hepatoblastoma, angiosarcoma, hepatocellular adenoma and hemangioma; (9) Vascular cell carcinoma, multimorphic cytocarcinoma, cytocarcinoma, osteoclastoid type, adenocarcinoma, adenocarcinoma, adenoepithelial carcinoma, mucus (colloidal) carcinoma, cystadenoma, acinar cell carcinoma, papillary carcinoma, small cell (oat cell) carcinoma, mixed cell type, carcinoma (NOS), undifferentiated carcinoma, cancer of the exocrine gland pancreas, including endocrine cell tumors and carcinoids occurring in Langerhans dormancytes; (10) Cancer of the salivary glands, including trilobe (samguli) cell carcinoma, adenocarcinoma (circumferential disease), adenocarcinoma, squamous cell carcinoma, carcinoma in polymorphic adenoma (malignant mixed tumors), mucosal epidermal carcinoma (well differentiated or low grade) and mucosal epidermal carcinoma (poorly differentiated or high grade); (11) Cancers of the stomach, including adenocarcinoma, papillary adenocarcinoma, coronary adenocarcinoma, mucous adenocarcinoma, ring cell carcinoma, adenoepithelial carcinoma, squamous cell carcinoma, small cell carcinoma, undifferentiated carcinoma, lymphoma, sarcoma and carcinoid tumors; And (12) cancer of the gastrointestinal tract, including cancer of the small intestine, including adenocarcinoma, lymphoma, carcinoid tumor, Kaposisarcoma, smooth fibroids, hemangioma, lipoma, neurofibromatosis and fibroma; (H) (1) cancer of the kidneys, including renal cell carcinoma, bellinian aggregate carcinoma, adenocarcinoma, papillary carcinoma, coronary carcinoma, granular cell carcinoma, transparent cell carcinoma (fresh cancer), sarcoma of the kidney and kidney blastoma; (2) Cancer of pyeloneph and ureters, including transitional cell carcinoma, papillary transitional cell carcinoma, squamous cell carcinoma and adenocarcinoma; (3) Cancer of the urethra, including transitional cell carcinoma, squamous cell carcinoma and adenocarcinoma; And (4) cancer of the urinary system, including cancer of the bladder, including orthogonal carcinoma, transitional urinary tract epithelial cell carcinoma, papillary transitional cell carcinoma, squamous cell carcinoma, adenocarcinoma, undifferentiation; (I) (1) (a) Osteogenesis: osteosarcoma; (b) Cartilage-formation: chondrosarcoma and mesenchymal cartilage sarcoma; (c) megaloblast tumors, malignant; (d) Ewing's sarcoma; (e) Vascular tumors: angioendothelioma, periangiocytoma and angiosarcoma; (f) connective tissue tumors: fibrosarcoma, adipose sarcoma, malignant mesenchyma and undifferentiated sarcoma; and (g) other tumors: cancer of the bone, including spinal cord and iliac panchamas; (2) Cancer of soft tissues, including alveolar soft sarcoma, angiosarcoma, epithelial sarcoma, extraneous cartilage sarcoma, fibrosarcoma, smooth myoma, liposarcoma, malignant fibrocytoma, malignant angioblastoma, malignant mesenchymoma, malignant Schwancelloma, rhabdomyomyoma, synovial sarcoma and sarcoma (NOS); (3) Cancer of the skull (osteomyoma, hemangioma, granuloma, yellowoma, deformable osteomyelitis), cancer of the meninges (meningioma, meningioma, glioma), cancer of the brain (star glioma, genital blastoma, glioma, ventricular membrane cytoma, seed cytoma (pineal glandular), polymorphic glioblastoma, rare glioma, Schwann cytoma, retinal blastoma, congenital tumors) and cancer of the spinal cord (neurofibromatosis, meningioma, glioma, sarcoma); (4) Myeloid leukemia (acute and chronic), acute lymphocyte leukemia, chronic lymphocyte leukemia, myeloproliferative disease, multiple myeloma; Blood cancers, including myelodysplastic syndrome), Hodgkin's disease and non-Hodgkin's lymphoma (malignant lymphoma); (5) (a) cancer of the thyroid gland, including papillary carcinoma (including those in the vesicle area), vesicular carcinoma, genital carcinoma and undifferentiated (anatomycinous) carcinoma; And (b) cancers of the endocrine system, including sympathetic blastoma, sympathetic neurocytema, malignant ganglion neuroma, ganglion sympathetic blastoma and neuroblastoma, including ganglion neuroma; (6) Cancer of the skin, including squamous cell carcinoma, spindle cell modification of squamous cell carcinoma, basal cell carcinoma, adenocarcinoma and malignant melanoma arising from the sebaceous or sebaceous glands; (7) (a) cancer of the conjunctiva, including carcinoma of the conjunctiva; (b) cancers of the blepharine, including basal cell carcinoma, squamous cell carcinoma, melanoma of the blepharium and sebum cell carcinoma; (c) cancers of the fistula, including adenocarcinoma, adenocarcinoma, carcinoma in polymorphic adenoma, mucous epidermal carcinoma and squamous cell carcinoma; (d) cancer of the uveal membrane, including spindle cell melanoma, mixed cell melanoma and epithelial cell melanoma; (e) cancers of the ophthalmology, including sarcoma of the ophthalmology, soft tissue tumors and sarcomas of the bone; And (f) may be selected from the group consisting of cancers of muscles, bones and soft tissues, including cancer of the eye, including retinal blastoma.

In addition, the cancer listed above may be a cancer in which the KRAS gene is mutated. In one embodiment of the present invention, the anticancer activity of the recombinant stabilized galectin-9 protein of the present invention was confirmed for colorectal, pancreatic, lung cancer and ovarian cancer as a carcinoma with the KRAS gene mutated.

The KRAS gene, which is one of the commonly mutated RAS genes in various cancers, causes functional changes in the product of the gene, the p21-RAS protein, when mutations occur in codons 12 and 13 of the KRAS gene. As a result, it promotes cell growth and division by transmitting growth signals excessively to the cytoplasm, contributing to the process of carcinogenesis. KRAS mutations are commonly found in human cancers, such as about 90% of pancreatic cancers, about 50% of colorectal cancers, and about 30% of non-small cell lung cancers.

In the present invention, the recombinant stabilized galectin-9 protein of the present invention or the polynucleotide encoding the same may be delivered by pharmaceutically acceptable carriers such as colloidal suspension, powder, saline, lipids, liposomes, microspheres, or nanospherical particles. These can form a complex with a vehicle or be associated therewith and can be delivered in vivo by using a delivery system well-known in the art such as lipids, liposomes, microparticles, gold nanoparticles, polymers, condensation reagents, polysaccharides, polyamino acids, dendrimers, saponins, adsorption enhancing substances or fatty acids.

In addition, examples of pharmaceutically acceptable carriers include lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate and mineral oil, which are commonly used in preparations, but the pharmaceutically acceptable carriers are not limited thereto. Further, in addition to the above components, a lubricant, a wetting agent, a sweetening agent, a flavoring agent, an emulsifying agent, a suspending agent, a preservative, and the like may be additionally included.

According to a desired method, the pharmaceutical composition of the present invention may be administered orally or parenterally (for example, intramuscularly, intravenously, intraperitoneally, subcutaneously, intradermally, or locally applied), and a dosage thereof may vary depending on states and weights of patients, the severity of a disease, drug forms, routes of the administration and time, but may be appropriately selected by those skilled in the art.

The pharmaceutical composition of the present invention is administered in a pharmaceutically effective amount.

As used herein, the term “pharmaceutically effective amount” refers to an amount sufficient to treat a disease with a reasonable benefit/risk ratio, which is applicable to medical treatments, and the effective dose level can be determined according to a patient's disease type, severity, drug activities, sensitivity to a drug, administration time, administration route, excretion rate, treatment duration, factors including concurrent drugs and other factors well-known in the medical field. The pharmaceutical composition according to the present invention may be administered as an individual therapeutic agent, may be used in combination with surgeries, hormone therapies, drug therapies and biological response modifiers, may be administered simultaneously, separately, or sequentially with the agents, and may be administered in a single does or multiple doses. It is important to administer an amount capable of obtaining the maximum effect with a minimum amount without side effects in consideration of all of the above factors, which can be easily determined by those skilled in the art.

Specifically, the effective amount of the pharmaceutical composition of the present invention may vary depending on a patient's age, sex, conditions, weight, absorption of the active ingredient into the body, inactivation rate, excretion rate, disease type, and drugs used in combination, and may be increased or decreased according to the administration route, the severity of obesity, the sex, the weight, the age, and the like.

In the present invention, the pharmaceutical composition may be formulated into various dosage forms selected from the group consisting of tablets, capsules, injections, troches, powders, granules, liquids (solutions), suspensions, oral solutions, emulsions, syrups, suppositories, vaginal tablets and pills, but not limited thereto, and may be formulated in appropriate formulations as needed. In addition, when formulating the composition, it is prepared using diluents or excipients such as fillers, bulking agents, binders, wetting agents, disintegrators, surfactants, etc., which are usually used.

Solid formulations for oral administration include tablets, pills, powders, granules, capsules, troches, and the like, and such solid formulations are prepared in one or more recombinant proteins of the present invention with at least one excipient, such as starch, calcium carbonate, sucrose or lactose or gelatin, and the like. In addition to simple excipients, lubricants such as magnesium stearate, talc are also used. Liquid preparations for oral administration include suspensions, oral solutions, emulsions or syrups, which are commonly used simple diluents such as water, liquid paraffin, various excipients, such as wetting agents, sweeteners, fragrances, preservatives, and the like.

Preparations for parenteral administration include sterile aqueous solutions, non-aqueous solvents, suspension solvents, emulsions, lyophilizing agents, suppositories, and the like.

As a non-aqueous solvent, a suspension solvent may be used as propylene glycol, polyethylene glycol, vegetable oil such as olive oil, injectable esters such as ethyl oleate, and the like. As a base of suppositories, Witepsol, Macrogol, Tween 61, cacao butter, laurin fat (oil), glycerol, gelatin, and the like may be used.

In addition, the present invention provides a pharmaceutical composition for preventing or treating cancer comprising a recombinant stabilized galectin-9 protein having an amino acid sequence represented by SEQ ID NO: 1 or a polynucleotide encoding the same and an anti-cancer agent as active ingredients of a complex, mixed, or combination agent; a method of preventing or treating cancer comprising administering a formulation comprising a recombinant stabilized galectin-9 protein having an amino acid sequence represented by SEQ ID NO: 1 or a polynucleotide encoding the same in a pharmaceutically effective amount and a formulation comprising an anticancer agent, to a subject in a complex, mixed, or combination manner; and use of pharmaceutical composition comprising a recombinant stabilized galectin-9 protein having an amino acid sequence represented by SEQ ID NO: 1 or a polynucleotide encoding the same, and anticancer agent in active ingredients of a complex, mixed, or combination agent for use in preventing or treating cancer.

In the present invention, examples of such anticancer agents may be, but are not limited to, cyclophosphamide, nimustine, gemcitabine, 5-fluorouracil (5-FU), tegafururacil (UFT), doxorubin, epirubicin, mitomycin, phleomycin, paclitaxel, docetaxel, cisplatin, oxaliplatin, Irinotecan, etoposide, Axitinib or Tarceva.

In the present invention, the concentration of the recombinant stabilized galectin-9 protein or the polynucleotide encoding the same may be from 0.01 to 100 mg/kg, and the concentration of the anticancer agent may be from 1 to 1000 mg/kg.

The amount of the active ingredient(s) in the pharmaceutical composition, which is a part of the medication, including a recombinant stabilized galectin-9 protein or a polynucleotide encoding the same, as well as an anticancer agent, can be appropriately selected based on factors such as the dosage form.

In the present invention, when the recombinant stabilized galectin-9 protein or the polynucleotide encoding the same, and anticancer agent are formulated into one single agent, the content of the recombinant stabilized galectin-9 protein or the polynucleotide encoding the same may be generally from about 0.01 to about 99.99 wt % for the entire formulation, specifically from about 0.01 to about 90 wt %, preferably from about 0.1 to about 90 wt %, and preferably from about 0.1 to about 90 wt %, more preferably from about 0.1 to about 80 wt %, more preferably from about 0.1 to about 70 wt %, and the anticancer agent is generally from about 0.01 to about 99.99 wt % for the entire formulation, specifically from about 0.01 to about 90 wt %, preferably from about 0.1 to about 80 wt %, more preferably from about 0.1 to about 70 wt %, more preferably from about 0.1 to about 60 wt %. On the other hand, in the case of being combined with one single formulation, the content ratio of the recombinant stabilized galectin-9 protein or the polynucleotide encoding the same and the anticancer agent in the medicine of the present invention may be combined in a 1:0.01 to 10 weight ratio. In addition, in the case of being combined with one single formulation, although the content of additives such as carriers and the like in the medicines of the present invention is variable, it may be generally from about 1 to about 99.00 wt % for the entire formulation, specifically from about 1 to about 90 wt %, preferably from about 10 to about 90 wt %, more preferably from about 10 to 80 wt %, and more preferably from about 10 to about 70 wt %.

In the present invention, when the recombinant stabilized galectin-9 protein or the polynucleotide encoding the same and anticancer agent are each formulated separately and used in combination, the content of the recombinant stabilized galectin-9 protein or the polynucleotide encoding the same in the formulation containing the recombinant stabilized galectin-9 protein or the polynucleotide encoding the same, may be generally from about 0.01 to about 99.99 wt % for the containing agent, specifically from about 0.1 to about 99.99 wt %, preferably from about 0.1 to about 90 wt %, more preferably from about 0.1 to about 80 wt %, and more preferably from about 1 to about 80 wt %. In addition, the content of the anticancer agent in the formulation containing the anticancer agent is generally from about 0.01 to about 99.99 wt % for the containing agent, specifically from about 0.1 to about 99.9 wt %, preferably from about 0.1 to about 90 wt %, and more preferably from about 0.1 to about 80 wt %. On the other hand, when the recombinant stabilized galectin-9 protein or the polynucleotide encoding the same and the anticancer agent are each formulated separately and used in combination, the content of additives such as carriers and the like is variable, for each containing agent may be generally from about 1 to 99.00 wt %, specifically from about 1 to about 90 wt %, preferably from about 10 to about 90 wt %, more preferably from about 10 to 80 wt %, more preferably from about 10 to about 70 wt %.

In addition, the present invention provides a complex, mixed, or combination kit for preventing or treating cancer, comprising a recombinant stabilized galectin-9 protein having an amino acid sequence represented by SEQ ID NO: 1, or a polynucleotide encoding the same and an anticancer agent as an active ingredient of a complex, mixed, or combination agent; and use of a complex, mixed or combination kit comprising a recombinant stabilized galectin-9 protein having an amino acid sequence represented by SEQ ID NO: 1, or a polynucleotide encoding the same and an anticancer agent as an active ingredient of a complex, mixed, or combination agent for use in preventing or treating cancer.

As for the combination kit cited above, the content and the content ratio of each component of the combination kit, and cancers will be described in the same manner as the pharmaceutical composition for cancer prevention or treatment in the preceding description, and so specific description for them will be cited in accordance with the preceding description.

In a specific embodiment of the invention, to confirm the anticancer activity of the recombinant stabilized galectin-9 protein having the amino acid sequence represented by SEQ ID NO: 1 and the anticancer activity of the complex, mixed, or combination agent of the recombinant protein and the anti-cancer agent, the protein was administered to colorectal, pancreatic cancer, lung cancer and ovarian cancer cells having a KRAS wild type or KRAS gene mutations, and the results showed inhibition of cell growth and induction of cell death. In addition, when the recombinant protein was co-administered with erlotinib to colorectal cancer cells with KRAS mutations, a synergistic effect on cell growth was observed.

In addition, when the recombinant protein or the anticancer agent was administered alone or when the recombinant protein and gemcitabine were administered in complex, mixed, or combination to a KRAS gene mutation-induced pancreatic cancer mouse model, a reduction in tumor size and an increase in survival rate were observed without any side effects such as weight loss. Furthermore, it was confirmed that the combined administration of the recombinant protein and anticancer agent showed a significant increase in efficacy compared to the administration of each agent alone.

In the present invention, the subject is a mammal in need of cancer treatment. In general, the target subject is a human cancer patient. In one embodiment of the invention, the subject may be a non-human mammal such as a non-human primate, an animal used in a model system (e.g., mice and rats used for screening, characterization and evaluation of drugs) and other mammals, such as rabbits, guinea pigs, hamsters, dogs, cats, chimpanzees, gorillas, and monkeys.

In one embodiment of the invention, the pharmaceutical composition may be used alone or in combination with surgery, hormone therapy, medication and biological response modulators for treatment of cancer patients.

In addition, the present invention provides a health functional food composition for preventing or improving cancer comprising a recombinant stabilized galectin-9 protein having an amino acid sequence represented by SEQ ID NO: 1 or a polynucleotide encoding the same, as an active ingredient; and use of a health functional food composition comprising a recombinant stabilized galectin-9 protein having an amino acid sequence represented by SEQ ID NO: 1 or a polynucleotide encoding the same as an active ingredient for use in preventing or improving cancer.

In the present invention, the recombinant stabilized galectin-9 protein and cancer are the same as described above, and the specific details will be provided in accordance with the preceding description.

As used herein, the term “improvement” refers to any actions that at least reduces parameters related to the condition being treated, for example, the severity of symptoms. In this case, the health functional food composition may be used before or after the onset of the disease in order to prevent or improve a bone disease, simultaneously with or separately from a drug for treatment.

On the other hand, since the recombinant stabilizing galectin-9 protein having the amino acid sequence represented by SEQ ID NO: 1 in the present invention has a cancer cell growth inhibition and apoptosis inducing effect, and it has been confirmed to have an effect of decreasing the size of cancer tissue and increasing the survival rate without side effects, the recombinant protein can be used as an active ingredient in a health functional food for cancer prevention or improvement.

In the health functional foods of the present invention, the active ingredient may be added to the food as it is or be used in conjunction with other food or food ingredients, and it may be used appropriately according to conventional methods. The mixed amount of the active ingredient may be suitably determined according to the purpose of its use (for prevention or improvement). In general, in the preparation of food or beverage, the health functional foods of the present invention may be added in an amount of preferably 15% or less by weight and preferably 10% or less for the raw material. However, in the case of long-term intake for health and hygiene purposes or for the purpose of health control, the amount may be below the above range.

The health functional food of the present invention may include other ingredients as essential ingredients without any specific limits other than containing the above active ingredients. For example, as in general beverages, various flavoring agents, natural carbohydrates, or the like may be included as additional ingredients. Examples of the above natural carbohydrates include monosaccharides such as glucose and fructose; disaccharides such as maltose and sucrose; and polysaccharides, for example, general sugars such as dextrin and cyclodextrin, and sugar alcohols such as xylitol, sorbitol, and erythritol. In addition to those described above, as flavoring agents, natural flavoring agents (thaumatin, stevia extract (for example, rebaudioside A and glycyrrhizin)) and synthetic flavoring agents (saccharin, aspartame, and the like) may be advantageously used. The ratio of the natural carbohydrate may be appropriately determined by the selection of those skilled in the art.

In addition to the above, the health functional food of the present invention may include various nutrients, vitamins, minerals (electrolytes), flavoring agents such as synthetic and natural flavoring agents, coloring agents and thickeners (cheese, chocolate, and the like), pectic acid and salts thereof, alginic acid and salts thereof, organic acids, protective colloidal thickeners, pH adjusters, stabilizers, preservatives, glycerin, alcohols, carbonates used in carbonated beverages, and the like. These components may be used independently or in combination, and the proportion of these additives may also be appropriately selected by those skilled in the art.

### EMBODIMENTS

Hereinafter, the present invention will be described in more detail through manufacturing examples and examples. However, the following preparation examples and examples are intended to help the understanding of the present invention and are not intended to limit the scope of rights of the present invention thereto.

### <Preparation Example 1> Preparation of Recombinant Stabilized Galectin-9 Protein

An expression vector including a gene that encodes the recombinant stabilized galectin-9 protein having the amino acid sequence of SEQ ID NO: 1 was prepared, and the expression vector was introduced into E. coli by a heat shock method. The recombinant protein was expressed by culturing E. coli in an LB medium including 50 μg/ml of kanamycin and adding arabinose when the absorbance at 600 nm reached 0.7 to induce the expression of the recombinant protein. Then, the cells induced to express the recombinant protein were lysed and filtered, and the target protein was captured by using a cation exchange method, an affinity column, and the like to obtain a highly purified recombinant stabilized galectin-9 protein in a high yield.

### <Example 1> Confirmation of In Vitro Anticancer Activity of a Recombinant Stabilized Galectin-9 Protein (sGal-9) and Anticancer Agents

<1-1> Confirming the Anti-Cancer Activity of the Recombinant Stabilized Galectin-9 Protein in Various Cancer Cell Lines

To investigate the anticancer activity of the recombinant stabilized galectin-9 protein having the amino acid sequence of SEQ ID NO: 1, cell growth and apoptosis were observed after treatment of the recombinant protein in cancer cell lines having a KRAS wild type or KRAS gene mutation.

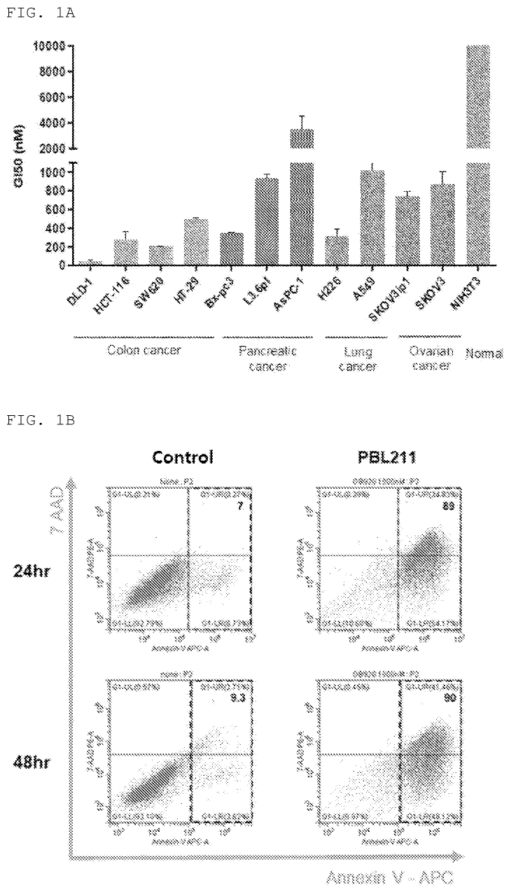

Specifically, to investigate the anticancer activity of the recombinant stabilized galectin-9 protein having the amino acid sequence of SEQ ID NO: 1, the protein was treated on cancer cell lines with KRAS wild-type or KRAS gene mutation, and the following cell lines were used: HT-29 for KRAS wild-type pancreatic cancer, DLD-1, HCT-116, and SW620 for KRAS gene-mutated colon cancer, Bx-pc3 for KRAS wild-type pancreatic cancer, L3.6pl and AsPC-1 for KRAS gene-mutated pancreatic cancer, H226 for KRAS wild-type lung cancer, A549 for KRAS gene-mutated lung cancer, SKOV3ip1 for KRAS wild-type ovarian cancer, and SKOV3 for KRAS gene-mutated ovarian cancer, and the cells were seeded at 5×10³ cells per well in a 96-well plate and cultured at 37° C. for one day, and then the recombinant protein was treated with concentrations of 3.2, 16, 80, 400, 2000, or 10,000 nM per well, and cultured for 72 hours. After 72 hours of incubation, the cell growth was confirmed using the SRB (sulforhodamine B) assay kit (Abcam, US) according to the manufacturer's instructions. NIH3T3 cells were used as negative controls (FIG. 1A).

In addition, DLD-1 cells, a colorectal cancer cell line with KRAS gene mutation, were seeded at a density of 1×10⁵ cells per well in a 6-well plates, cultured for 1 day at 37° C. and the recombinant protein from <Preparation Example 1> was then added to each well at a concentration of 1,000 nM, and the cells were further cultured for either 24 or 48 hours. After washing the cultured cells twice with cold PBD, the cells were stained using Annexin-APC and 7-AAD apoptosis kit (Biolegend, US) according to the manufacturer's instructions, and apoptosis was analyzed using a flow cytometer (CytoFLEX, Beckmancoulter) (FIG. 1B).

As a result, as shown in FIGS. 1A and 1B, treatment with the recombinant protein inhibited cell growth and induced apoptosis in KRAS wild-type or mutant colorectal, pancreatic, lung cancer and ovarian cancer cells compared to the negative control group.

<1-2> Confirming the Anti-Cancer Activity of Recombinant Stabilized Galectin-9 Protein in Colorectal Cancer Cell Lines with KRAS Gene Mutations

To investigate the anti-cancer activity of the recombinant stabilized galectin-9 protein having the amino acid sequence of SEQ ID NO: 1, the proliferation of cancer cells by colony formation assay was analyzed by treating KRAS mutant colorectal cancer cell lines with the recombinant protein.

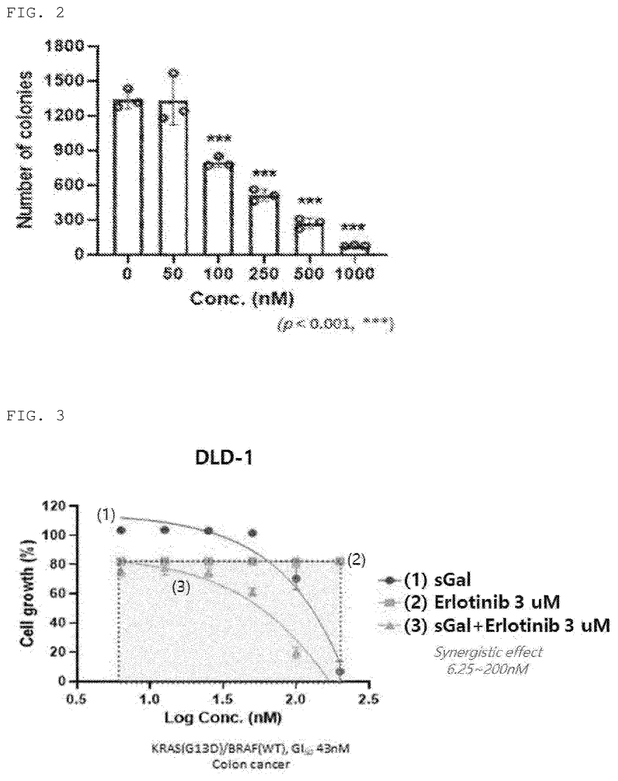

Specifically, DLD-1 cells, a colorectal cancer cell line with KRAS gene mutation, were seeded in 6-well plates at 5×10³ cells per well in a medium containing 3% v/v Matrigel, and then treated with the recombinant protein from <Preparation Example 1> at concentrations of 0, 50, 100, 250, 500, 1000 nM and incubated for 10 days. Then, after washing with phosphate-buffered saline, colonies were stained with a solution of 0.05% crystal violet, 1% formaldehyde, and 1% methanol in phosphate-buffered saline for 20 minutes at room temperature. The plate was then washing with distilled water and dried at room temperature for 16 hours. The number of colonies was measured by capturing images of the stained and dried plates and analyzing the images (FIG. 2).

As a result, as shown in FIG. 2, it was confirmed that the colony formation ability of cancer cells decreased as the concentration of the recombinant protein treatment increased, indicating that the recombinant protein inhibited the proliferation of cancer cells in a concentration-dependent manner.

<1-3> Confirmation of In Vitro Anticancer Activity of Recombinant Stabilized Galectin-9 Protein and Anti-Cancer Drug

To investigate the anticancer activity of a recombinant stabilized galectin-9 protein having an amino acid sequence represented by SEQ ID NO: 1, in combination with an anticancer agent, KRAS mutant colorectal cancer cell lines were treated with the recombinant protein and erlotinib (brand name: Tarceva), an EGFR inhibitor, and cell growth was evaluated.

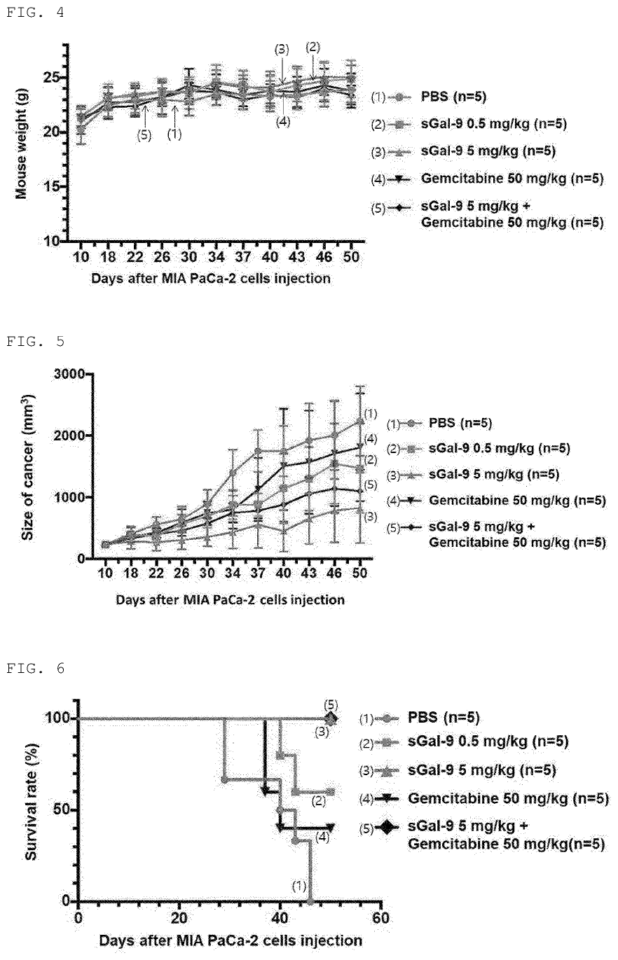

Specifically, KRAS mutant colorectal cancer cells, DLD-1 cells, were seeded in 96-well plates at 5×10³ per well, and treated with a fixed concentration of 3 uM erlotinib alone or in combination with the recombinant stabilized galectin-9 protein prepared according to <preparation example 1>, at varying concentrations of 3, 2, 16, 80, 400, 2000, or 10000 nM. After 72 hours of incubation, cell growth was assessed using the sulforhodamine B (SRB) assay kit (abcam, US) following the manufacturer's protocol (FIG. 3).

As a result, as shown in FIG. 3, it was confirmed that the synergistic effect of cell growth inhibition was shown in cells treated with recombinant protein and erlotinib in combination compared to cells treated with each of the recombinant protein and erlotinib alone.

### <Example 2> Confirmation of In Vivo Anticancer Activity of Recombinant Stable Galectin-9 Protein (sGal-9) and the Anticancer Agent

<2-1> Identification of Weight Changes by Administration of Recombinantly Stabilized Galectin-9 Protein and/or Gemcitabine in Pancreatic Cancer-Induced Mouse Models

To investigate the anticancer activity of the recombinant stabilized galectin-9 protein having the amino acid sequence SEQ ID NO: 1, KRAS gene mutant pancreatic cancer cell line MIA PaCa-2 cells were transplanted and grown for two weeks to form pancreatic tumor tissue subcutaneously in mice, and the mice were then administered with the recombinant protein, and changes in body weight were monitored.

Specifically, the experiments were conducted in accordance with the Guide for the Care and Use of Laboratory Animals (NRC), and the mice were managed accordingly. After collecting mice with an average pancreatic cancer size of 200 nm 3, they were randomly classified into 5 group.

The experimental groups were classified into five groups (5 mice per group) as follows: control group (PBS), low-dose recombinant stabilized galectin-9 protein administration group (seal-9 0.5 mg/kg), high-dose recombinant stabilized galectin-9 protein administration group (seal-9 5 mg/kg), gemcitabine group (Gemcitabine 50 mg/kg), and combination of seal-9 and gemcitabine (seal-9 5 mg/kg+Gemcitabine 50 mg/kg). The mice were administered with seal-9 the given concentrations twice a week and/or gemcitabine once a week by intraperitoneal injection for 50 days, while monitoring changes in body weight (FIG. 4).

As a result, as shown in FIG. 4, no significant changes in body weight were observed in the control group, low-dose seal-9 treatment group, high-dose seal-9 treatment group, and seal-9 and gemcitabine combination treatment group, indicating that there were no side effects of seal-9 monotherapy or combination therapy with an anticancer agent.

<2-2> Confirmation of Changes in Cancer Tissue Size by Administration of Recombinant Stabilized Galectin-9 Protein and/or Gemcitabine in Pancreatic Cancer-Induced Mouse Models

To investigate the anticancer activity of the recombinant stabilized galectin-9 protein having the amino acid sequence of SEQ ID NO: 1, the KRAS gene mutant pancreatic cancer (MIA PaCa-2) mouse model was established by using the same method described in Example <2-1> above, and seal-9 and/or gemcitabine were administered into the mice for 50 days and the change in the size of the pancreatic cancer tissue was evaluated using magnetic resonance imaging (MRI) (FIG. 5).

As a result, as shown in FIG. 5, the study found that the size of pancreatic cancer tissue decreased significantly in the seal-9 low- and high-dose groups compared to the control group, and seal-9 showed a superior effect at lower doses than conventional chemotherapy. In addition, it was confirmed that the combined administration of seal-9 and gemcitabine showed a superior synergistic effect compared to the administration of gemcitabine alone.

<2-3> Confirmation of Changes in Survival of Recombinant Stabilized Galectin-9 Protein and Gemcitabine in Pancreatic Cancer-Induced Mouse Models

To investigate the anticancer activity of the recombinant stabilized galectin-9 protein having an amino acid sequence of SEQ ID NO: 1, the same method as described in Example 2-1 was used to administer seal-9 and/or gemcitabine to a KRAS gene mutated pancreatic cancer (MIA PaCa-2) mouse model, and the survival rate was monitored for 50 days (FIG. 6). In this case, a pancreatic cancer tissue size of 1700 mm3 or more was considered as the time of death.

As a result, as shown in FIG. 6, it was confirmed that the seal-9 low- and high-dose groups had superior survival rates. In addition, it was observed that the combined administration of seal-9 and gemcitabine showed a superior therapeutic effect compared to the administration of gemcitabine alone.

### INDUSTRIAL APPLICABILITY

The recombinant stabilized galectin-9 protein according to the present invention exhibits the effect of inhibiting cell growth and inducing apoptosis of cancer cells, and exhibits the effect of decreasing the size and increasing survival rate of cancer tissue without side effects in cancer-causing animal models, and may be useful as an active ingredient in a composition for cancer prevention or treatment, since it exhibits synergistic anticancer activity when administered in complex, mixed, or in combination with an anticancer agent.

1. A method of treating cancer, the method comprising administering to a subject a pharmaceutically effective amount of a recombinant stabilized galectin-9 protein having an amino acid sequence represented by SEQ ID NO: 1 or a polynucleotide encoding the same.

2. The method of treating cancer according to claim 1, wherein the recombinant stabilized galectin-9 protein comprises deletion of the first amino acid residue from the N-terminus of the amino acid sequence represented by SEQ ID NO: 1.

3. The method of treating cancer according to claim 1, wherein the cancer is characterized by having KRAS mutation.

4. The method of treating cancer according to claim 1, wherein the cancer is selected from the group consisting of (A) (1) orthogonal conduit carcinoma (DCIS) (cotton carcinoma, ideogram, nipple, micropapilloma), invasive conduit carcinoma (IDC), tubular carcinoma, mucus (colloidal) carcinoma, papillary carcinoma, incarnate carcinoma and conduit carcinoma, including inflammatory carcinoma; (2) lobular carcinoma, including orthogonal lobular carcinoma (LCIS) and invasive lobular carcinoma; and (3) breast cancer, including paget's disease of the nipples; (B) (1) cervical epithelial tumors (Grade I), cervical epithelial tumors (Class II), intracervical epithelial tumors (Class III) (orthogonal squamous cell carcinoma), keratinized squamous cell carcinoma, non-keratinous squamous cell carcinoma, wart-shaped carcinoma, orthogonal adenocarcinoma, orthogonal adenocarcinoma, endometrial adenocarcinoma, transparent cell adenocarcinoma, adenoepithelial carcinoma, adenocarcinoma, adenocarcinoma, small cell carcinoma and undifferentiated carcinoma; (2) Cancer of the uterine body, including endometrial carcinoma, adenocarcinoma, adenocarcinoma (adenocarcinoma with squamous epithelium), adenocarcinoma (mixed adenocarcinoma and squamous cell carcinoma, mucous adenocarcinoma, serous adenocarcinoma, transparent cell adenocarcinoma, squamous cell adenocarcinoma and undifferentiated adenocarcinoma; (3) Cancer of the ovaries, including serous cystadenoma, serous cystadenoma, mucous cystadenoma, mucous cyst adenoma, endometrial tumor, endometrial adenocarcinoma, transparent cell tumor, transparent cell cyst adenoma and unclassified tumor; (4) vaginal cancers, including squamous cell carcinoma and adenocarcinoma; And (5) tumors in the vulva epithelial (Class I), tumors in the vulva epithelial (Class II), tumors in the vulva epithelial (Class III) (orthogonal squamous cell carcinoma); Cancers of the female reproductive system, including cancer of the vulva, including squamous cell carcinoma, wart-shaped carcinoma, paget's disease of the pubic gland, adenocarcinoma (NOS), basal cell carcinoma (NOS) and Bartholin adenocarcinoma; (C) (1) cancer of the penis, including squamous cell carcinoma; (2) Cancer of the prostate, including adenocarcinoma of the prostate, sarcoma and transitional cell carcinoma; (3) Cancers of the male reproductive system, including cancers of the testicles, including normal-hematoma tumors, abnormal hematoma tumors, teratomas, embryonic carcinoma, yolk cyst tumors and chorionic carcinoma; (D) sarcoma (angiosarcoma, fibrosarcoma, rhabdomyosarcoma, liposarcoma), myxoma, rhabdomyomyoma, fibroma, lipoma and teratoma of the heart system; (E) cancers of the respiratory system, including squamous cell carcinoma of the larynx, primary pleural mesothelioma and squamous cell carcinoma of the pharynx; (F) squamous cell carcinoma (epidermal carcinoma), variants of squamous cell carcinoma, spindle cell carcinoma, small cell carcinoma, carcinoma, carcinoma, carcinoma of other cells, carcinoma of intermediate cell type, complex oat cell carcinoma, adenocarcinoma, granular adenocarcinoma, papillary adenocarcinoma, bronchial alveolar carcinoma, mucous forming solid carcinoma, megaloblastic carcinoma, cytocarcinoma, transparent cell carcinoma, and cancer of the lungs, including sarcoma; (G) (1) cancer of the Vater bulge, including primary adenocarcinoma, carcinoid tumors and lymphoma; (2) Cancer of the tract, including adenocarcinoma, squamous cell carcinoma and melanoma; (3) Cancer of the extrahepatic bile ducts, including orthogonal carcinoma, adenocarcinoma, adenocarcinoma, intestinal type, mucous adenocarcinoma, transparent cell adenocarcinoma, ring cell carcinoma, adenoepithelial carcinoma, squamous cell carcinoma, small cell (oats) carcinoma, undifferentiated carcinoma, carcinoma (NOS), sarcoma and carcinoid tumors; (4) Orthogonal adenocarcinoma, adenocarcinoma, mucous adenocarcinoma (colloidal type; mucous carcinoma greater than 50%), ring cell carcinoma (ring cells greater than 50%), squamous cell (epidermis-shaped) carcinoma, adenoepithelial carcinoma, small cell (oat cell) carcinoma, undifferentiated carcinoma, carcinoma (NOS), sarcoma, lymphoma and cancer of the colon and rectum, including carcinoma; (5) Cancer of the esophagus, including squamous cell carcinoma, adenocarcinoma, smooth myoma and lymphoma; (6) Cancer of the gallbladder, including adenocarcinoma, adenocarcinoma, bowel type, adenoepithelial carcinoma, orthogonal carcinoma, carcinoma (NOS), transparent cell adenocarcinoma, mucous adenocarcinoma, papillary adenocarcinoma, ring cell carcinoma, small cell (oat cell) carcinoma, squamous cell carcinoma and undifferentiated carcinoma; (7) Cancer of the lips and oral cavity, including squamous cell carcinoma; (8) Cancer of the liver, including liver cancer (hepatocellular carcinoma), bile duct carcinoma, hepatoblastoma, angiosarcoma, hepatocellular adenoma and hemangioma; (9) Vascular cell carcinoma, multimorphic cytocarcinoma, cytocarcinoma, osteoclastoid type, adenocarcinoma, adenocarcinoma, adenoepithelial carcinoma, mucus (colloidal) carcinoma, cystadenoma, acinar cell carcinoma, papillary carcinoma, small cell (oat cell) carcinoma, mixed cell type, carcinoma (NOS), undifferentiated carcinoma, cancer of the exocrine gland pancreas, including endocrine cell tumors and carcinoids occurring in Langerhans dormancytes; (10) Cancer of the salivary glands, including trilobe (samguli) cell carcinoma, adenocarcinoma (circumferential disease), adenocarcinoma, squamous cell carcinoma, carcinoma in polymorphic adenoma (malignant mixed tumors), mucosal epidermal carcinoma (well differentiated or low grade) and mucosal epidermal carcinoma (poorly differentiated or high grade); (11) Cancers of the stomach, including adenocarcinoma, papillary adenocarcinoma, coronary adenocarcinoma, mucous adenocarcinoma, ring cell carcinoma, adenoepithelial carcinoma, squamous cell carcinoma, small cell carcinoma, undifferentiated carcinoma, lymphoma, sarcoma and carcinoid tumors; and (12) cancer of the gastrointestinal tract, including cancer of the small intestine, including adenocarcinoma, lymphoma, carcinoid tumor, Kaposisarcoma, smooth fibroids, hemangioma, lipoma, neurofibromatosis and fibroma; (H) (1) cancer of the kidneys, including renal cell carcinoma, bellinian aggregate carcinoma, adenocarcinoma, papillary carcinoma, coronary carcinoma, granular cell carcinoma, transparent cell carcinoma (fresh cancer), sarcoma of the kidney and kidney blastoma; (2) Cancer of pyeloneph and ureters, including transitional cell carcinoma, papillary transitional cell carcinoma, squamous cell carcinoma and adenocarcinoma; (3) Cancer of the urethra, including transitional cell carcinoma, squamous cell carcinoma and adenocarcinoma; And (4) cancer of the urinary system, including cancer of the bladder, including orthogonal carcinoma, transitional urinary tract epithelial cell carcinoma, papillary transitional cell carcinoma, squamous cell carcinoma, adenocarcinoma, undifferentiation; (I) (1) (a) Osteogenesis: osteosarcoma; (b) Cartilage-formation: chondrosarcoma and mesenchymal cartilage sarcoma; (c) megaloblast tumors, malignant; (d) Ewing's sarcoma; (e) Vascular tumors: angioendothelioma, periangiocytoma and angiosarcoma; (f) connective tissue tumors: fibrosarcoma, adipose sarcoma, malignant mesenchyma and undifferentiated sarcoma; and (g) other tumors: cancer of the bone, including spinal cord and iliac panchamas; (2) Cancer of soft tissues, including alveolar soft sarcoma, angiosarcoma, epithelial sarcoma, extraneous cartilage sarcoma, fibrosarcoma, smooth myoma, liposarcoma, malignant fibrocytoma, malignant angioblastoma, malignant mesenchymoma, malignant Schwancelloma, rhabdomyomyoma, synovial sarcoma and sarcoma (NOS); (3) Cancer of the skull (osteomyoma, hemangioma, granuloma, yellowoma, deformable osteomyelitis), cancer of the meninges (meningioma, meningioma, glioma), cancer of the brain (star glioma, genital blastoma, glioma, ventricular membrane cytoma, seed cytoma (pineal glandular), polymorphic glioblastoma, rare glioma, Schwann cytoma, retinal blastoma, congenital tumors) and cancer of the spinal cord (neurofibromatosis, meningioma, glioma, sarcoma); (4) Myeloid leukemia (acute and chronic), acute lymphocyte leukemia, chronic lymphocyte leukemia, myeloproliferative disease, multiple myeloma; Blood cancers, including myelodysplastic syndrome), Hodgkin's disease and non-Hodgkin's lymphoma (malignant lymphoma); (5) (a) cancer of the thyroid gland, including papillary carcinoma (including those in the vesicle area), vesicular carcinoma, genital carcinoma and undifferentiated (anatomycinous) carcinoma; And (b) cancers of the endocrine system, including sympathetic blastoma, sympathetic neurocytema, malignant ganglion neuroma, ganglion sympathetic blastoma and neuroblastoma, including ganglion neuroma; (6) Cancer of the skin, including squamous cell carcinoma, spindle cell modification of squamous cell carcinoma, basal cell carcinoma, adenocarcinoma and malignant melanoma arising from the sebaceous or sebaceous glands; (7) (a) cancer of the conjunctiva, including carcinoma of the conjunctiva; (b) cancers of the blepharine, including basal cell carcinoma, squamous cell carcinoma, melanoma of the blepharium and sebum cell carcinoma; (c) cancers of the fistula, including adenocarcinoma, adenocarcinoma, carcinoma in polymorphic adenoma, mucous epidermal carcinoma and squamous cell carcinoma; (d) cancer of the uveal membrane, including spindle cell melanoma, mixed cell melanoma and epithelial cell melanoma; (e) cancers of the ophthalmology, including sarcoma of the ophthalmology, soft tissue tumors and sarcomas of the bone; And (f) may be selected from the group consisting of cancers of muscles, bones and soft tissues, including cancer of the eye, including retinal blastoma.

5. The method of treating cancer according to claim 1, wherein the method further comprises administering a pharmaceutically acceptable carrier.

6. The method of treating cancer according to claim 1, wherein the recombinant stabilized galectin-9 protein having an amino acid sequence represented by SEQ ID NO: 1 or the polynucleotide encoding the same is formulated for oral administration, intramuscular administration, intravenous administration, intraperitoneal administration, subcutaneous administration, intradermal administration, or topical administration.

7. The method of treating cancer according to claim 1, wherein the recombinant stabilized galectin-9 protein having an amino acid sequence represented by SEQ ID NO: 1 or polynucleotide encoding the same is formulated into a formulation selected from the group consisting of a tablet, a capsule, an injection, a troche, a powder, a granule, a solution, a suspension, an oral solution, an emulsion, a syrup, a suppository, a vaginal tablet and a pill.

8. A method of treating cancer, the method comprising administering to a subject a formulation comprising a pharmaceutically effective amount of a recombinant stabilized galectin-9 protein having an amino acid sequence represented by SEQ ID NO: 1 or a polynucleotide encoding the same; and a formulation comprising an anticancer agent, in a complex, mixed or combination manner.

9. The method of treating cancer according to claim 8, wherein the anticancer agent is selected from the group consisting of Cyclophosphamide, Nimustine, Gemcitabine, 5-Fluorouracil (5-FU), Tegafur/uracil (UFT), Doxorubin, Epirubicin, Mitomycin, Phleomycin, Paclitaxel, Docetaxel, Cisplatin, Oxaliplatin, Irinotecan, Etoposide, Axitinib, and Tarceva.

10. The method of treating cancer according to claim 8, wherein the combination ratio of the recombinant stabilized galectin-9 protein having an amino acid sequence represented by SEQ ID NO: 1 or the polynucleotide encoding the same; and the anticancer agent is 1:0.01 to 1:10 by weight.

11. (canceled)

12. (canceled)

13. (canceled)

14. A method of preventing cancer comprising administering to a subject a pharmaceutically effective amount of a recombinant stabilized galectin-9 protein having an amino acid sequence represented by SEQ ID NO: 1 or a polynucleotide encoding the same.

15. (canceled)

16. (canceled)

17. (canceled)

18. (canceled)

19. (canceled)
